(19) United States
(12) Patent Application Publication
(10) Pub. No.: US 2021/0161713 A1
(43) Pub. Date: Jun. 3, 2021

(54) DRAINAGE DEVICE AND METHODS

(71) Applicant: UCL BUSINESS LTD., London (GB)

(72) Inventors: Yann BOUREMEL, London (GB); Peng Tee KHAW, London (GB)

(73) Assignee: UCL Business Ltd., London (GB)

(21) Appl. No.: 17/044,558

(22) PCT Filed: Apr. 2, 2019
(86) PCT No.: PCT/GB2019/050949

§ 371 Date: Oct. 1, 2020

(30) Foreign Application Priority Data
Apr. 3, 2018 (GB) 1805439.5

(51) Int. Cl. A61F 9/007; B29C 48/11; B29C 48/00; B23K 26/38

(52) U.S. Cl. CPC A61F 9/00781; B29C 48/11; B29C 48/0022; B29L 2031/7546; A61F 2240/001; A61F 2230/0008; A61F 2250/0091; B23K 26/38

(57) ABSTRACT

A drainage device for use in an eye to drain aqueous humour so as to reduce intraocular pressure or for treating glaucoma. The device has a multi-lumen tube having a first end, a second end opposite the first end, and a plurality of lumen extending between the first end and the second end. At least one of the lumen is sealed at the first end. A flow through the multi-lumen tube is adjusted by forming at least one aperture open in one of the lumen through a wall of the tube and/or sealing at least one aperture open in one of the lumen. The tube has a longitudinal axis through the first end and the second end, and an outer surface extending between the first end and the second end. A cross-section perpendicular to the longitudinal axis has a non-circular shape at the outer surface with an aspect ratio of at least 3:1.

### FIELD OF THE INVENTION

The present invention relates to a drainage device and methods for use in the treatment of glaucoma.

### BACKGROUND OF THE INVENTION

Glaucoma is an irreversible chronic optic neuropathy with characteristic optic nerve head changes and visual field defects. In the eye aqueous fluid is produced by the ciliary body and reaches the anterior chamber formed between the iris and the cornea through the pupil. In a normal eye, the aqueous is removed through the trabecular meshwork. There the aqueous passes through Schlemm's canal and through veins which merge with blood-carrying veins and into venous circulation. Intraocular pressure is maintained in the eye by the intricate balance of secretion and absorption or outflow of the aqueous in the manner described above. Glaucoma results from excessive build-up of aqueous fluid in the anterior chamber producing an increase in intraocular pressure (IOP), which is the major modifiable risk factor associated with glaucoma.

Raised intra-ocular pressure (IOP) can be treated with medication, laser or surgery. Glaucoma drainage devices (GDDs) are useful adjuncts in surgical management but are usually reserved for patients following failed glaucoma filtration surgery or in patients with conditions that respond poorly to trabeculectomy such as neovascular, uveitic and paediatric glaucoma. GDD implantation requires a high level of surgical skill and experience and can require surgical times of 45 to 90 minutes. Additionally, the ability to control the lowering of IOP and to adjust it after surgery using conventional GDDs is poor. Most contemporary GDDs do not achieve the lower level of IOP required to minimise glaucomatous progression.

One of the first known GDD devices was the Molteno®, as described in WO 2005/092260, which comprises a circular polypropylene plate with an inner ridge defining a primary draining region, an optional outer ridge defining a secondary draining region and a hole in the inner ridge to connect a drainage tube. The ridges are intended to prevent post-operative hypotony.

Other GDD devices include those described in WO2010/054035, which comprise a single lumen drainage tube and a plate wherein the tube has a hoop strength such that the tube collapses after insertion into an incision and expands later. The drainage tube is intended to act as a flow restrictor, increasing outflow of aqueous fluid over time.

A further design is described in US20040215126. This device incorporates a drainage tube, a plate and a one-way valve intended to respond to IOP. This device is popular in the US because it can be inserted into a patient and a follow-up is not needed for at least three months.

The majority of current market GDD products rely on fibrosis to control pressure within the eye. However, if there is too much fibrosis the devices will fail. Equally, if there is too little fibrosis there is a risk of post-operative hypotony.

The present inventors have therefore identified a need to provide a GDD capable of controlling IOP through tube flow rate rather than fibrosis.

### SUMMARY OF THE INVENTION

A first aspect of the invention provides a drainage device for use in an eye to drain aqueous humour so as to reduce intraocular pressure, the device comprising a multi-lumen tube having a first end, a second end opposite the first end, and a plurality of lumen extending between the first end and the second end, wherein at least one of the lumen is sealed at the first end.

The invention of the first aspect is advantageous in that one or more apertures can be provided at locations along the length of the sealed lumen to create a fluid path between the second end and the aperture, the location of the aperture defining the length of the fluid path and therefore the flow resistance and resultant pressure drop along the fluid path can be selected. By fine tuning the pressure drop the drainage device can be tailored or ‘titrated’ to adjust the intra-ocular pressure (IOP) to individual patient requirements. This can be particularly advantageous for patients who require an IOP of less than 10 mmHg, as the IOP can be reduced in small increments, which allows the risk of hypotony (TOP of less than 5 mmHg) to be reduced or avoided.

The device may further comprise at least one aperture open in said at least one of the lumen through a sidewall of the tube and located along the length of the tube between the first end and the second end, wherein the at least one aperture fluidly connects the second end of the tube to outside the tube through said lumen.

The aperture(s) may be formed in the tube to achieve a desired fluid flow pressure drop through the tube. Alternatively, the aperture(s) may be formed in the tube and one or more of the apertures may be selectively closed prior to use to achieve a desired fluid flow pressure drop through the tube.

The tube may be marked at points where apertures may be formed. The markings can be used to indicate suitable points for creating apertures, which may be selected depending on the desired fluid flow pressure drop through the tube. The markings may be provided in the form of selectively thinned areas of the tube material, for example, the markings may be provided as etchings in the tube material. Reducing the thickness of the tube material may provide advantages in facilitating apertures being formed at the marked points.

A distance from the second end of the tube to the aperture may be selected to provide predetermined resistance to fluid flow through the device.

The drainage device may have a plurality of the apertures. For example, a plurality of the lumen may each have at least one of the apertures discretely through the sidewall. Alternatively, said at least one of the lumen has a plurality of the apertures spaced along the length of the tube.

The said at least one of the lumen may have an internal diameter selected to provide predetermined resistance to fluid flow through the device.

The tube may be flexible. In particular it may be advantageous for the tube to be sufficiently flexible to follow around the curvature or globe of the eye. Whilst the tube may be sufficiently flexible to follow the curvature of the eye, it is typically in a substantially linear configuration. In other words the tube is not significantly bent laterally or formed into a loop.

A second aspect of the invention provides a drainage device for use in an eye to drain aqueous humour so as to reduce intraocular pressure, the device comprising a flexible multi-lumen tube having a first end, a second end opposite the first end, a longitudinal axis through the first end and the second end, a plurality of lumen extending between the first end and the second end, and an outer surface extending between the first end and the second end, wherein a cross-section perpendicular to the longitudinal axis has a non-circular shape at the outer surface.

The invention of the second aspect is advantageous in that the non-circular shape enables the tube to have flexibility without being prone to kinking. This makes the tube easier to handle. The non-circular shape may also better enable the tube to seal with a cut formed in tissue (i.e. tube shape conformal with cut profile) than a circular shape would.

The cross-section shape at the outer surface may be substantially oval or elliptical. In embodiments of the invention the cross-section shape at the outer surface may be an ellipse. The cross-section shape at the outer surface may have an aspect ratio (width to height) of at least 3:1, or at least 4:1, or at least 5:1. Preferably the cross-section shape at the outer surface may have an aspect ratio (width to height) of at least 6:1, or at least 7:1, or at least 8:1.

The aspect ratio of the cross-section shape at the outer surface may provide particular advantages including, but not limited to, reducing or preventing sideways/lateral movement and/or rotation or twisting of the tube around its longitudinal axis. The aspect ratio of the cross-section shape at the outer surface can also provide improved pressure distribution as the pressure of the tube on surrounding tissues and cells in the patient's eye is distributed across the width of the tube and hence over a larger area than would be the case with a round tube of a similar volume.

The tube may be anisotropic in bending about two axes each perpendicular to the longitudinal axis. The tube may have a width and a height, and the plurality of lumen may be spaced in the width dimension, and the tube may have a greater bending flexibility in a plane including the height dimension than in a plane including the width dimension. The tube may be malleable in one direction and stiff in another.

The following statements may apply to the first and/or the second aspects of the invention.

The drainage device may be a glaucoma drainage device (GDD).

Each lumen may have a diameter of between approximately 40 microns to approximately 200 microns, preferably between approximately 45 microns to approximately 110 microns.

Each lumen may have a substantially constant cross section along the length of the tube.

The lumens may be positioned non-symmetrically within the tube. For example, each lumen may be arranged so as to be closer to the surface of the tube that is uppermost when the device is in use. The thickness of the sidewall between each lumen and the surface of the tube that is lowermost when the device is in use may be at least 4 or at least 5 times the thickness of the sidewall between each lumen and the surface of the tube that is uppermost when the device is in use. The thickness of the sidewall between each lumen and the uppermost surface of the device may be about 30 microns or less, or about 20 microns or less. In embodiments of the invention the thickness of the sidewall between each lumen and the uppermost surface of the device may be about 12 microns to about 20 microns.

The tube length may be between approximately 5 mm to approximately 30 mm, preferably between approximately 5 mm to approximately 20 mm, more preferably between approximately 8 mm to approximately 15 mm.

The tube width may be between approximately 0.5 mm to approximately 3 mm, preferably between approximately 1 mm to approximately 2 mm.

The tube may have a maximum height of approximately 500 microns or less, preferably approximately 300 microns or less, more preferably approximately 200 microns or less.

Two or more of the lumen may have different internal diameters.

One or more of the lumen may have a substantially circular cross section.

The tube may include biocompatible and/or biostable material. The biocompatible and/or biostable material may be provided as a coating on a tube substrate material.

The tube may include at least one of plastics material and silicone.

The tube may have a sidewall having a thickness of between approximately 5 microns to approximately 200 microns, preferably between approximately 20 microns to approximately 100 microns.

The tube may include transparent or translucent material. Lower opacity may be beneficial to be able to observe the aperture(s) through an opposite side of the device.

Each lumen may be valveless and/or filterless. In embodiments of the invention the lumen do not include a membrane and/or internal protrusions. The pressure drop of fluid flow through the device may be bidirectional.

The first end of the tube may have a bevelled edge. The bevelled edge may improve insertability through tissue. In embodiments of the invention the first end of the tube may be tipped (rounded). The tipping the first end of the tube may reduce damage to surrounding tissues when the tube is implanted. The second end of the tube may also be tipped if convenient.

The drainage device may further comprise generally planar extensions projecting from the tube intermediate the first and second ends. The generally planar extensions may be in the form of stabilising ‘wings’. The extensions may prevent or restrict rotation of the tube about its longitudinal axis when in use. The extensions may have a length in the tube longitudinal direction of less than 5 mm. The region of the tube having the extensions may be free of apertures. The extensions may be located at a region of the tube intended to be embedded in tissue when in use.

At least one aperture may be located between the generally planar extensions and the first end.

The drainage device may further comprise a plate adapted to locate on the eye. The plate may be adapted to prevent or restrict rotation of the tube about its longitudinal axis when in use. The plate may be used as an alternative to the generally planar extensions.

The tube may be adapted to be secured to the plate and the at least one aperture may be open adjacent the plate.

The drainage device, including any one or more of the tube, the planar extensions and/or the plate may comprise a biocompatible and/or bioactive coating. A bioactive coating typically comprises a drug or a compound, such as a small molecule or peptide, that has a biological effect on surrounding tissue when the device is in use. When the device is in use the drug or compound may be released from the bioactive coating over time. Bioactive coatings may include drugs or compounds that are anti-fibrotic (e.g., anticancer agents such as mytomycin-c or 5-flurouracil) metalloprotease (MMP) inhibitors (such as ilomastat, lenalidomide or tranilast), anti-inflammatory (such as steroids), non-steroidal anti-inflammatory agents, and/or anti-angiogenic. Biocompatible coatings may include polymer coatings, e.g. comprising phosphorylcholine (PC).

A further aspect of the invention provides a method of manufacturing a drainage device for use in an eye to drain aqueous humour so as to reduce intraocular pressure, comprising providing a multi-lumen tube having a first end, a second end opposite the first end, a plurality of lumen extending between the first end and the second end, and adjusting a flow through the multi-lumen tube by forming at least one aperture open in one of the lumen through a wall of the tube and/or sealing at least one aperture open in one of the lumen. The method may be used to form the tube of the first or second aspects of the invention.

The step of forming the aperture may comprise forming the aperture through a sidewall of the tube.

At least one of the lumen may be sealed at the first end to provide an end wall, and the step of forming the aperture may comprise forming the aperture through the end wall of the sealed first end.

The step of sealing the aperture may comprise either closing an open first end of the lumen or closing an aperture through a sidewall of the tube.

The aperture is preferably formed by laser cutting. A YAG or Argon laser may be used, for example. Alternatively, the aperture may be formed by puncturing.

The multi-lumen tube may be made by extrusion, drawing or injection moulding. In particular, the tube may be formed by extruding a multi-lumen preform through a die, stretching the preform in the longitudinal direction to reduce the lumen diameter, and cutting to a desired tube length. The extruded material may be a plastics material. Stretching the extruded preform may achieve small diameter lumen not achievable with a directly extruded product.

Moulding the device from silicon material may be advantageous in that the generally planar extensions or ‘wings’ can be co-moulded with the tube. Extruding the tube means that the extensions need to be attached later.

A further aspect of the invention provides a method for treating glaucoma or controlling intraocular pressure in a patient's eye, the method comprising positioning the first end of the drainage device according to the first and/or second aspects in the anterior chamber of the patient's eye, and positioning the second end of the drainage device in the subconjunctival space of the patient's eye.

The method may further comprise opening one or more apertures in one or more of the lumens to control the flow rate of aqueous humour through the drainage device. The apertures may be opened prior to insertion of the device into the eye or after insertion of the device into the eye. Opening apertures after insertion of the device allows IOP to be adjusted on an ongoing basis while the device is in situ. This can be advantageous for patients in which IOP increases over time, e.g., due to an increase in resistance from the bleb. Apertures may be opened to increase the flow rate of aqueous humor through the device as required. For example, IOP of patients may be monitored at regular intervals. In the event that the IOP increases so as to be above a threshold one or more apertures may be opened to drop the IOP to the desired value for that patient. As described above, apertures may be opened using a laser, such as a YAG laser, which are commonly available in ophthalmic departments.

The patient is preferably a mammal, including a human, and may be a paediatric or geriatric patient.

Optionally, once inserted into the eye the device may be secured using a suture. However, in embodiments of the invention wherein the device comprises planer extensions or a plate no suturing may be needed.

The device of the present invention can be inserted quickly and easily through an incision, which may be formed using a stepped profile blade. The blade may allow for a single pass incision to be used. The ease of insertion of the device of the present invention may reduce the level of surgical skill and amount of surgical time required to implant the device. For example, it may be possible to implant the device in as little as 10 minutes. This means that a greater number of patients may be able to benefit from the device, meaning that the device can have a greater impact on world blindness than conventional GDDs.

A further aspect of the invention provides a method for preparing a drainage device according to the first and/or second aspects for surgery, the method comprising: comparing an intraocular pressure measurement obtained from a patient with a threshold to calculate the required drop in intraocular pressure, and opening one or more apertures in one or more of the lumens to control the flow rate of aqueous humour through the drainage device and provide the required drop in intraocular pressure.

In embodiments of the invention the device will provide an IOP of about 5 mmHg to about 22 mmHg, preferably about 7 mmHg to about 15 mmHg. In embodiments of the invention the device provides an IOP of about 10 mmHg.

Without being bound by theory, while conventional GDDs have a failure rate of around 10% per year, the inventors believe that the device of the present invention may have a survival period of up to 10 years due to the ability to tailor or titrate IOP of patients. This will provide improved quality of life for patients and reduce healthcare costs by reducing the need for further surgical intervention.

A yet further aspect of the invention provides a kit comprising a drainage device according to the first and/or second aspects and forceps. The forceps are preferably complimentary to the drainage device. The kit may additionally comprise a knife, which may have a blade comprising a stepped profile and/or an inserter.

### BRIEF DESCRIPTION OF THE DRAWINGS

Embodiments of the invention will now be described with reference to the accompanying drawings, in which:

FIG. 1 shows a glaucoma drainage device (GDD) connecting the anterior chamber of the eye to a bleb in the subconjuctival space;

FIGS. 2 to 9 show various views of the GDD;

FIGS. 10(a)-(c) show various examples of the GDD with apertures in different locations to adjust the pressure drop though the GDD;

FIGS. 11 and 12 show the pressure drop for the examples of FIGS. 10(a)-(c);

FIGS. 13(a)-(f) show the change in pressure drop though an aperture of different diameters;

FIGS. 14(a)-(d) show various alternative cross sections for the GDD; and

FIG. 15 shows a plate for use with the GDD.

FIG. 16 shows a GDD divided into three parts: A, B and C.

FIG. 17 shows Finite-Element Analysis (FEA) simulation of a GDD with force applied at the back of the tube when held in place at the wings before bending (a), and after bending (b).

FIG. 18 shows a simulation of a conjunctival map for mitomycin-c (MMC) application during trabeculectomy surgery (Khaw et al. Dev. Opthalmol. 2017, 59:15-35).

FIG. 19 shows deflections and associated Von Mises stress of conjunctival flaps. (a)-(b): 1 mm indentation by 1 mm wide; (c)-(d): 0.5 mm indentation by 0.5 mm wide; (e)-(f): 0.25 mm indentation by 0.25 mm wide; and (g)-(h): 0.125 mm wide. Each indentation is 2.5 mm long.

FIG. 21 shows (a) elliptical tubes ranging from eccentricity of 0 (circular tube) to 0.98 (an example of the GDD described herein) with (b): the definition of height (H) and width (b) of elliptical tube.

FIG. 22 shows a model of an incision shape when a circular tube is inserted through it.

FIG. 23 shows a cross-section of tubes of different eccentricity with a 0.500 mm incision shown at the outer boundary. All dimensions are in mm.

FIG. 24 shows computational fluid dynamics analysis of the initial set-up (a) and flow pathlines (b) for a circular tube of external diameter 0.2 mm, length 3 mm and a lumen diameter of 0.05 mm. The incision is 0.2 mm high and 0.5 mm wide and 3 mm long. The flow rate was fixed at 2 μl/min and the pressure drop was less than 0.1 mmHg.

FIG. 25 shows computational fluid dynamics analysis of the initial set-up (a) and flow pathlines (b) for an elliptical tube (H=0.2 mm, b=0.438 mm and length of 3 mm) with a lumen diameter of 0.05 mm. The incision is 0.2 mm high, 0.5 mm wide and 3 mm long. The flow rate was fixed at 2 μl/min and the pressure drop was around 3 mmHg.

FIG. 26 shows computational fluid dynamics analysis of the initial set-up (a) and flow pathlines (b) for an elliptical tube (H=0.2 mm, b=0.492 mm and length of 3 mm) with a lumen diameter of 0.05 mm. The incision is 0.2 mm high, 0.5 mm wide and 3 mm long. The flow rate was fixed at 2 μl/min and the pressure drop was around 5 mmHg.

FIG. 27 shows computational fluid dynamics analysis of pathlines around an elliptical rod (H=0.2 mm, b=0.492 mm and length of 3 mm) inserted into an incision of 0.2 mm high, 0.5 mm wide and 3 mm long. The flow rate was fixed at 2 μl/min and the pressure drop was in excess of 400 mmHg.

FIG. 28 shows (a) variation of the second moment area ratio Iₑₓ/Iᵣₓ, with b/H and (b) example of deflection of the GDD along the y axis.

FIG. 29 shows (a) variation of the second moment area ratio I_{ey}/I_{ry}, with b/H and (b) example of deflection of the GDD along the x axis.

FIG. 30 shows Von Mises Stresses of a circular cylinder of diameter 0.2 mm (a) and elliptical (0.2 mm by 1 mm) when bent with a force of 1 mN.

FIG. 31 shows a schematic of a tube being deflected upward where exiting from the incision into the subconjunctival space (a) and the corresponding Finite-Element Analysis (FEA) model for a circular tube of diameter 0.2 mm (b) and an elliptical tube of height 0.2 mm and width 1 mm (c) deflected upward by 1 mm.

FIG. 32 shows Von Mises Stress along the tube corresponding to the deflections mentioned in FIG. 31 for the circular tube in (a-b) and elliptical tube (c-d).

### DETAILED DESCRIPTION OF EMBODIMENT(S)

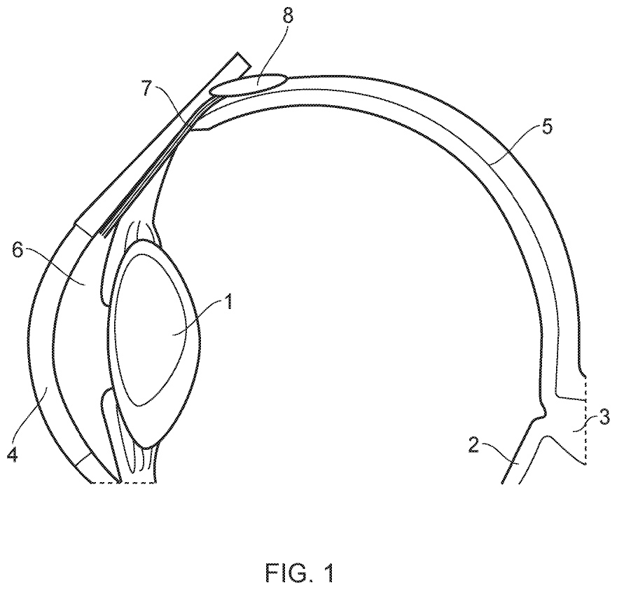

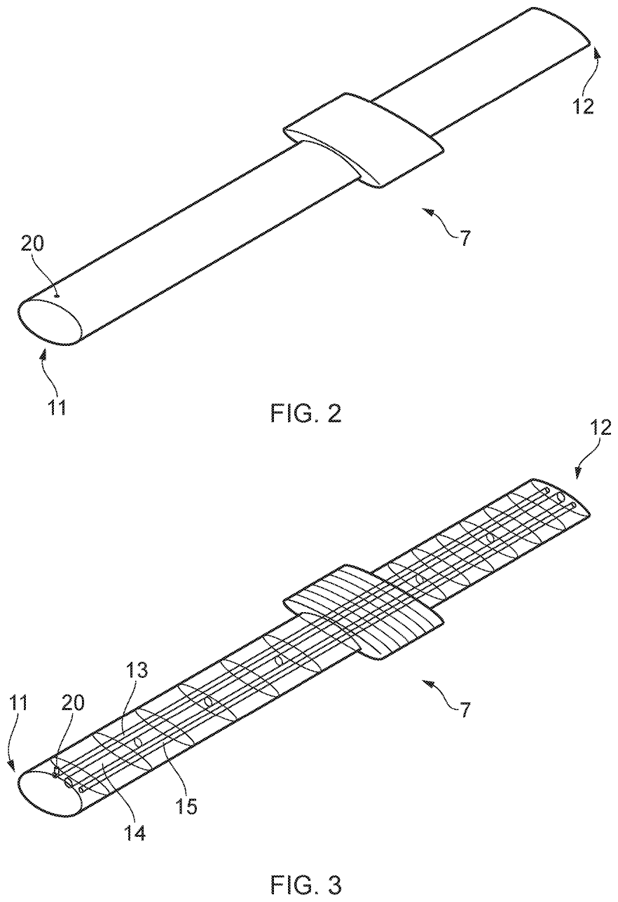

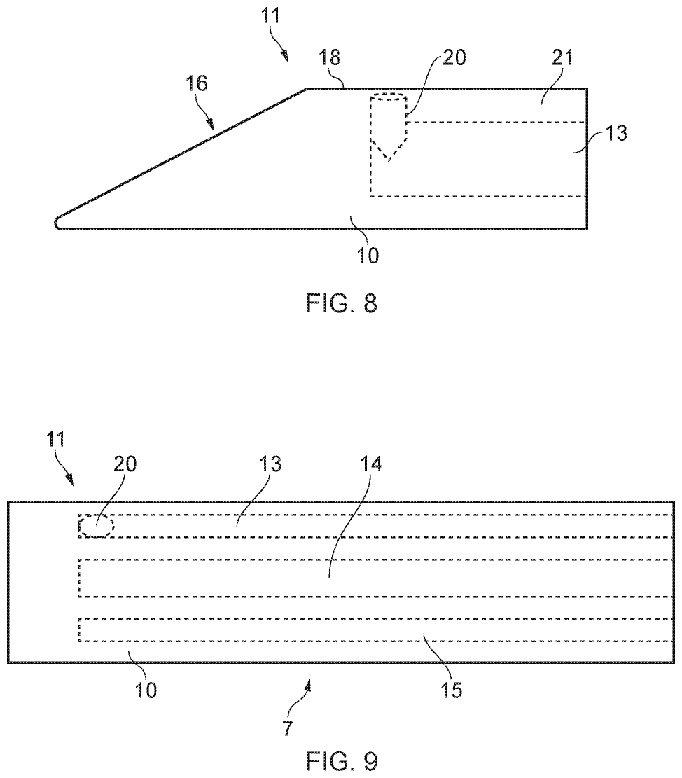

FIG. 1 illustrates schematically an eye, e.g. of a human, showing the lens 1, retina 2, optic nerve 3, cornea 4, sclera 5 and anterior chamber 6. A glaucoma drainage device (GDD) 7 is used to fluidly connect the anterior chamber 6 to a bleb 8 in the subconjunctival space following trabeculectomy so as to lower the intra-ocular pressure (IOP). The GDD 7 in accordance with a first embodiment is shown in detail in FIGS. 2 to 9.

### Shape of the Device

As shown in FIG. 2, the GDD 7 is a triple-lumen tube having a first end 11 and a second end 12 opposite the first end. FIG. 3 is an ‘X-ray’ type image showing three lumens 13, 14, 15 extending from the first end to the second end. When in use, the first end 11 is for locating in the anterior chamber 6 and the second end is for locating in the subconjunctival space to discharge aqueous humour into the bleb 8.

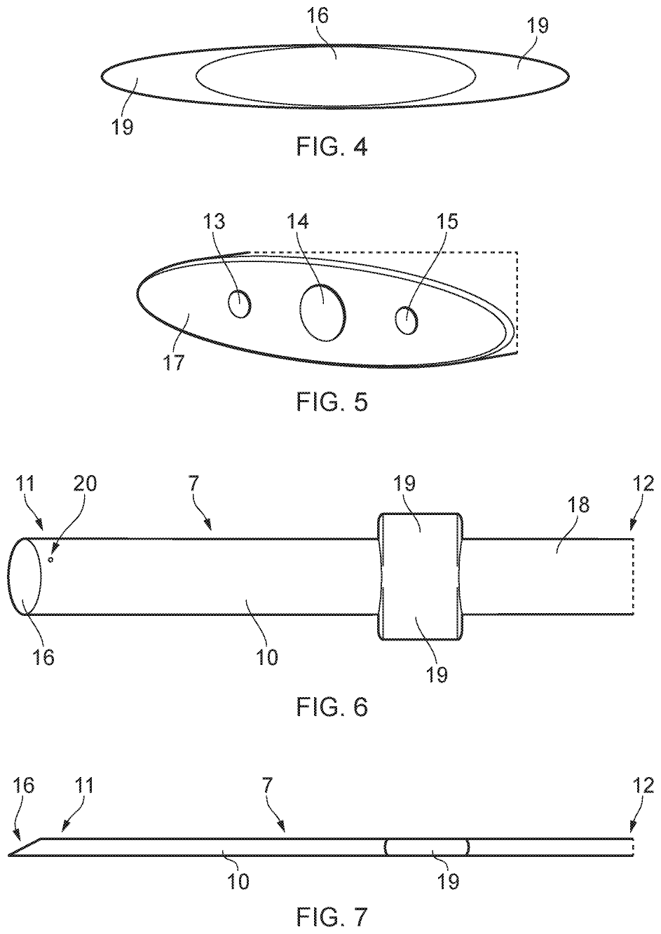

The tube has a first end face 16 at the first end 11, and a second end face 17 at the second end 12. As best shown in FIG. 4, the first end face 16 is closed (sealed) to all three lumens 13, 14, 15 and, as best shown in FIG. 5, the second end face 17 is open to all three lumen 13, 14, 15.

The lumen 13, 14, 15 extend in the longitudinal direction of the tube 10. The central lumen 14 has a larger diameter than the outer lumen 13, 15.

The tube 10 has an outer surface 18 extending between the first and second end faces 16, 17. The tube 10 has sidewalls between the outer surface 18 and the respective lumen 13, 14, 15. The outer surface 18 has an oval shaped cross section, having an aspect ratio (width to height) of at least 3:1.

As best shown in FIGS. 6, 8 and 9, one lumen 13 has an aperture 20 open through the sidewall 21 near the first end 11 so as to fluidly connect the second end 12 to the outside of the tube 10 through the lumen 13. The location of the aperture 20 is selected to provide a fluid path between the aperture 20 and the open second end of the lumen 13 to provide a predetermined pressure drop across the GDD 7, as will be described in detail later. The aperture 20 may be formed by lasering the exterior of the tube, for example, as will be described in detail later.

Adopting an oval cross-section configuration helps to accommodate the three lumens 13, 14, 15 of different sizes while maintaining a small tube 10 height by placing the lumens 13, 14, 15 laterally next to each other. A round tube where all lumens are located in the centre of the tube would increase the sidewall thickness of the central lumen 14. This has implications for lasering the exterior of the lumen to create the aperture 20.

The oval cross-section also helps reduce any lateral movement when in-situ. A circular cross section tube would require the same force to deflect longitudinally and laterally. However, the oval shape makes it relatively more difficult to deflect the tube in bending laterally compared to longitudinally, indeed the ratio between lateral and longitudinal deflection is (width/height)². In the embodiment illustrated in FIGS. 2 to 9, the oval tube 10 with height=0.2 mm and width=0.9 mm, requires 20.25 times more force to deflect the tube laterally than longitudinally.

Stabilising generally planar extensions (or ‘wings’) 19 project from the outer surface 18 intermediate the first and second ends 11, 12. In the illustrated embodiment the wings 19 are located 4.5 mm behind the first end 11 of the tube 10. The ‘wings’ 19 or more generally a part to minimise tube movement (notches along the tube 10 could also be possible) help the GDD 7 to stay in place behind the limbus by preventing the tube 10 to slide in the anterior chamber 6.

The first end 11 is bevelled in the longitudinal direction. In the illustrated embodiment of FIGS. 2 to 9 the bevel angle is approximately 27° to give a bevel length of 0.4 mm or twice the height of the tube 10. The force to insert the GDD 7 into the conjunctiva is reduced when the first end 11 of the tube 10 is bevelled.

Finally, to minimise the damage of the tube 10 to the surrounding tissues when the tube is implanted, the first end 11 of the tube 10 is tipped (rounded). The second end 12 of the tube may also be tipped if convenient.

### Flow Resistance of the Device

The GDD 7 controls the pressure drop through its lumens 13, 14, 15. A classic Hagen-Poiseuille law describes pressure drop through circular tube or lumen as shown in Equation 1:

$$δ P = \frac{128 υ Lq}{π {D}^{4}} \qquad ( 1 )$$

with δP is the pressure drop though the lumen, v, the dynamic viscosity of the fluid, q, the flow rate and D, the diameter of the lumen and L, the length of the lumen. The resistance of the lumen is independent of the flow rate and is only defined by geometrical parameters and the dynamic viscosity of the fluid. Equation (1) can be rewritten in terms of resistance (R) as P=Q×R with R defined as:

$$R = \frac{128 vL}{π {D}^{4}} \qquad ( 2 )$$

The diameter of lumen 13 is selected to provide a pressure drop of no less than 5 mmHg at 2 μl/min and a temperature of 36.7° C. Taking a tolerance for the lumen diameter of 3 μm, over a distance of 7.4 mm (to take into account the bevel length of 0.4 mm+a maximum plugged length of 0.2 mm), the maximum lumen diameter was calculated to be 57 μm such as to give a pressure drop of 5 mmHg at 2 μl/min and a temperature of 36.7° C. Any diameter above that value has been calculated to give a pressure drop lower than 5 mmHg. The diameter of the lumen 13 (and lumen 15) is therefore 54+/−3 μm.

If the IOP builds up in time due to different reasons such as an increase of resistance from the bleb 8, the GDD 7 offers the possibility to open lumen 15 by lasering above the tube 10 creating another aperture 20 to form a fluid path from the device exterior to the second end 12. When opening the lumen 15 at the same location as aperture 20 in lumen 13, the pressure drop is exactly 50% of that using aperture 20 in lumen 13 alone as each resistance acts in parallel.

The middle lumen 14 has a diameter of 110 μm. The middle lumen diameter is selected to reduce the pressure drop through the GDD 7 as much as possible without having an excessively thin sidewall 21 around the middle lumen 14. Ideally the GDD 7 has near zero flow resistance when the middle lumen 14 is opened. For example, middle lumen 14 may be opened in the case that the IOP of the patient should be reduced as much as possible using the GDD 7.

The choice of the diameter of the middle lumen 14 may be selected to ensure the pressure drop though the GDD 7 relatively low and ideally below 0.5 mmHg for 2 μl/min. With the middle lumen diameter of 110 μm, a pressure drop of approximately 0.3 mmHg is achieved when all three lumens 13, 14, 15 are opened at the first end 11 of the tube 10.

### Resistance Adjustment Potential

The GDD 7 offers the possibility to alter the resistance of the device by lasering multiple apertures along a single lumen or by lasering more than one lumens or a combination of both. When lasering along a single lumen, the resistance of the device is proportional to the length of the tube as shown in Equation (2) (all other parameters being equal) and therefore the pressure drop is entirely affected by the length of the lumen. When opening more than one lumen in parallel, the resistance of N lumen adds up following Equation (3):

$${R}_{tot = {\sum }_{1}^{N}} \frac{1}{\frac{1}{{R}_{N}}} \qquad ( 3 )$$

The final pressure drop given by the GDD 7 is δP=QRₜₒₜ. Altering the resistance of the GDD 7 opens up minute modifications of the pressure drop given by the GDD. The GDD offers the possibility to open any of the three lumens 13, 14, 15 up to 3.5 mm from the first end 11 of the device (for a minimum lumen length of 4.5 mm).

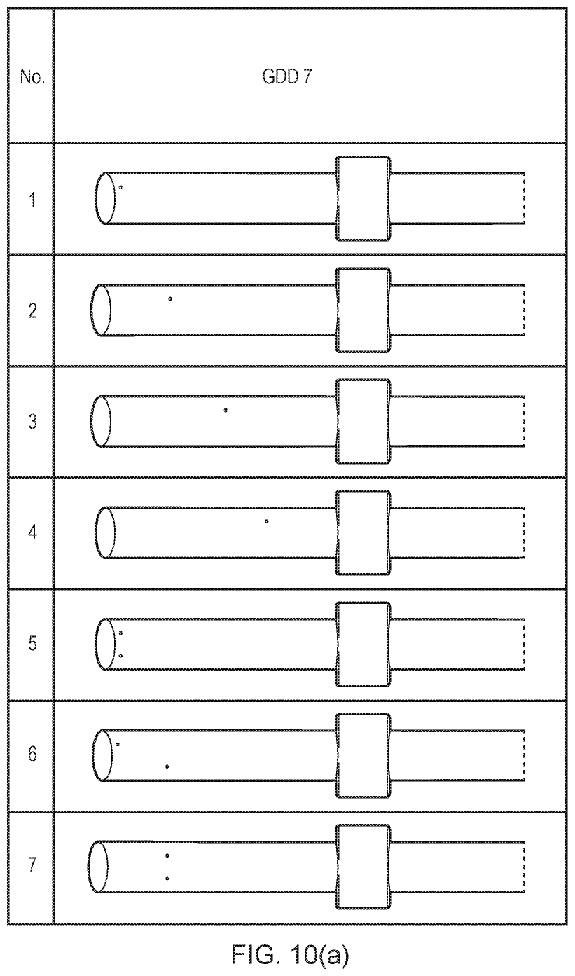

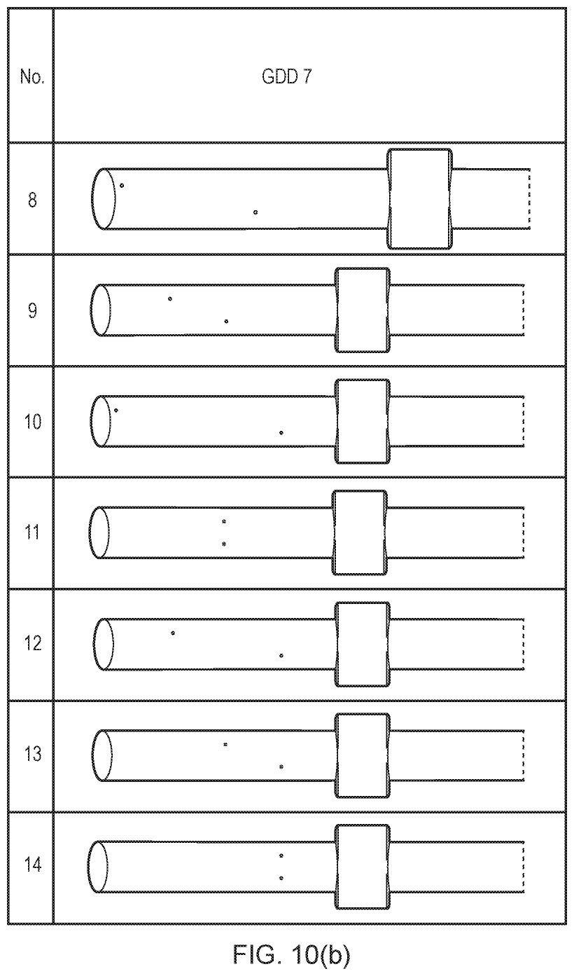

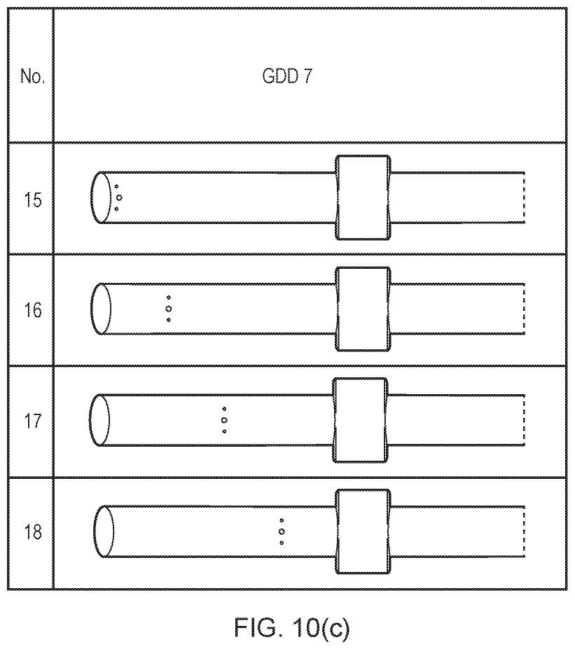

FIGS. 10(a) to (c) illustrate 18 variants of the GDD 7 each numbered 1-18 and each having a different configuration of the aperture(s) 20 open in one or more of the lumen 13, 14, 15.

Table 1 shows the resistance of the device as well as the pressure drop (at 2 μl/min) for locations lasered 1 mm apart along each lumen.

TABLE 1
Resistance and pressure drop at 2 μl/min of the GDD 7 when
lasered along and across each lumen.

| No. | Resistance | δP (mmHg) at 2 μl/min |
|---|---|---|
| 1. | ≈100% | ≈6.3 |
| 2. | ≈87% | ≈5.5 |
| 3. | ≈73% | ≈4.6 |
| 4. | ≈60% | ≈3.8 |
| 5. | ≈50% | ≈3.2 |
| 6. | ≈46% | ≈2.9 |
| 7. | ≈43% | ≈2.7 |
| 8. | ≈42% | ≈2.7 |
| 9. | ≈40% | ≈2.5 |
| 10. | ≈38% | ≈2.4 |
| 11. | ≈37% | ≈2.3 |
| 12. | ≈35% | ≈2.2 |
| 13. | ≈33% | ≈2.1 |
| 14. | ≈30% | ≈1.9 |
| 15. | ≈0.05% | ≈0.3 |
| 16. | ≈0.05% | ≈0.3 |
| 17. | ≈0.04% | ≈0.2 |
| 18. | ≈0.03% | ≈0.2 |

Position 1 corresponds to a lumen length L of 7.5 mm, position 2 to L=6.5 mm, position 3 to L=5.5 mm and position 4 to L=4.5 mm. The resistance of each lumen is decreased by a maximum of 40% when opening the lumen at position 4, but the combination of lumens working in parallel achieve a larger pressure reduction up to approximately 0.02 mmHg at 2 μl/min and 36.7° C. when each lumen are opened at the position 4. Positions 1, 2, 3, and 4 are hypothetical locations, but any locations in between each of the positions are also possible giving an infinite control of the resistance of the GDD 7 and hence the IOP of the patient.

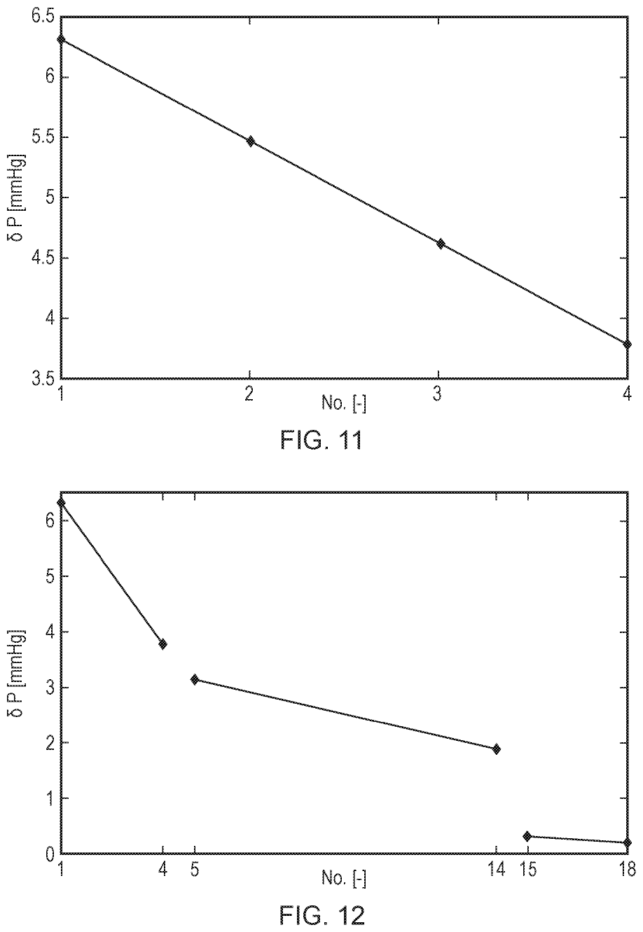

FIG. 11 shows the pressure drop at 2 μl/min referring to the different configurations reported in FIGS. 10(a) to (c) and Table 1 along one of lumens 13 or 15. FIG. 12 shows the pressure drop at 2 μl/min referring to the different configurations reported in FIGS. 10(a) to (c) and Table 1 along all three lumens 13, 14, 15. The curves in FIG. 12 are coded corresponding to the pressure drop decrease when opening one lumen shown in solid line, dashed line to two open lumens and dot-dashed line to three open lumens. It is important to note that any pressure drop δP value shown by the three curves is achievable. It is intended that the GDD 7 could provide any continuous resistance decrease from 100% to 0%. However for the selected diameters of the illustrated embodiment there are some gaps.

Opening the first lumen (13 or 15) enables the decrease of the resistance of the device by up to 40%; opening the two side lumens (13 and 15) enables the decrease of the resistance by between approximately 50 and 70%; opening the two side lumens and the middle lumen (13, 14 and 15) enables the decrease of the resistance by between approximately 99.5 and 99.7%. It is worth noting that opening one side lumen and the middle lumen will also decrease the resistance between approximately 99.5 and 99.7%.

The GDD 7 offers the possibility to alter the resistance and hence the pressure drop by simply lasering along each lumen 13, 14, 15. The GDD 7 may include silicon or plastics material such as polyurethane. The apertures may be formed by laser cutting. A YAG or Argon laser may be used, for example. Alternatively, the aperture may be formed by puncturing each lumen as necessary to achieve the desired flow rate.

The multi-lumen tube 10 may be made by extruding a multi-lumen preform through a die, stretching the preform in the longitudinal direction to reduce the lumen diameter, and cutting to a desired tube length. The extruded material may be a plastics material. Stretching the extruded preform may achieve small diameter lumen not achievable with a directly extruded product. Suitable plastics may include polycarbonate, phosphorycholine hydrogel, polyether block amide, polycarbonate based polyurethanes, aliphatic based polyurethanes and nylon, for example. A biocompatible material, or biocompatible coating, may be used.

Moulding the device from silicon material may be advantageous in that the generally planar extensions or ‘wings’ 19 can be co-moulded with the tube. Extruding the tube means that the extensions need to be attached later.

Lasering along the GDD 7 gives the ability to alter the flow resistance. Lumens are lasered from the top, therefore Poiseuille law does not fully apply along the apertures 20 (holes) created by lasering as they have approximately the same width and length and a correction need to be added. Flows through holes may be considered as the combination of two flows, the Sampson flow and the Poiseuille flow. For a typical hole of radius equal to 50 μm and a length equal to 30 μm, the Sampson component is twice the Poiseuille flow, therefore it is important to consider both parameters when looking at flows through holes. It is expected that the minimum sidewall 21 thickness may be around 25 μm or less.

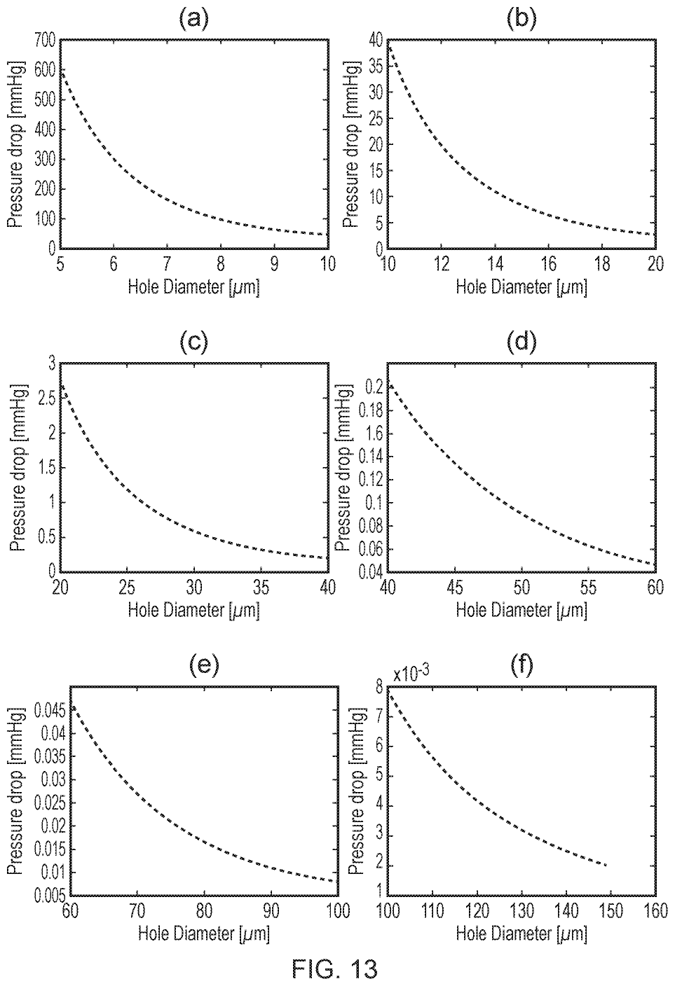

FIGS. 13(a) to (f) shows the pressure drop for increasing hole diameter, i.e. 5 to 10 μm reported in FIG. 13(a), 10 to 20 μm in FIG. 13(b), 20 to 40 μm in FIG. 13(c), 40 to 60 μm in FIG. 13(d), 60 to 100 μm in FIG. 13(e) and 100 to 160 μm in FIG. 13(f). It can be seen that for an aperture 20 diameter above approximately 26 μm the pressure drop introduced by the aperture is less than half of 1 mmHg.

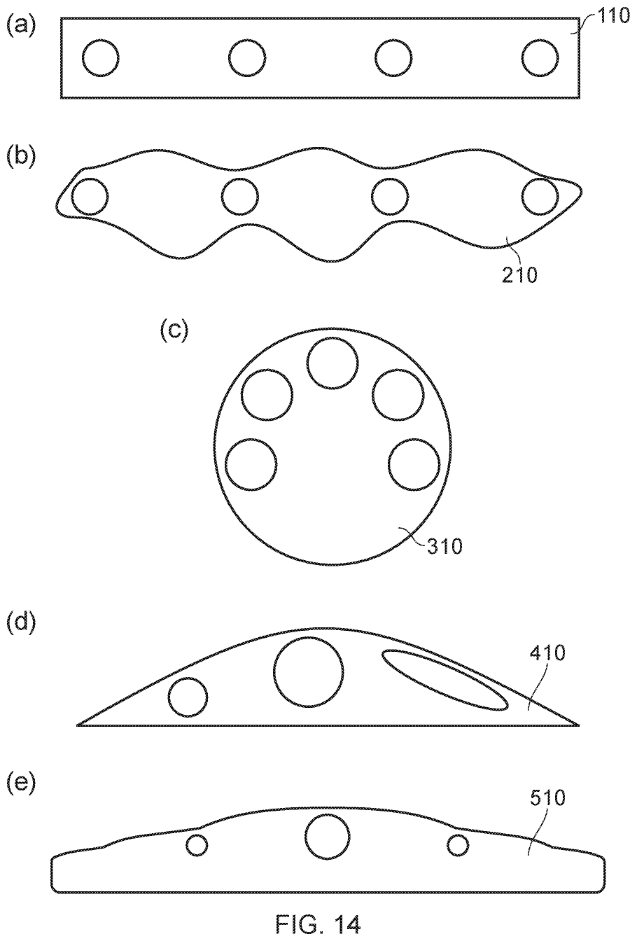

Whilst in the illustrated embodiment above the GDD 7 has an oval shape cross-section it is useful to consider other GDD cross-section shapes that could accommodate more than one lumen as shown in FIGS. 14(a)-(e). FIG. 14(a) shows a tube 110 with rectangular cross-section with four lumens. FIG. 14(b) shows a tube 210 with a section having variable thickness and four lumens located adjacent respective upper and lower troughs between respective upper and lower ridges. FIG. 14(c) shows a tube 310 having a circular cross section with five lumens arranged radially around only an upper portion of the tube so as to be substantially equidistant from the centre of the section. FIG. 14(d) shows a tube 410 having a crescent shaped cross-section with three lumens, two of which are circular of different diameters and one of which is elliptical. It will be appreciated that the invention may employ a variety of cross sections and a variety of lumens of different number, shape, size, etc. FIG. 14(e) shows a tube 510 having an aspect ratio (width to height) of at least about 7:1 and having three circular lumens arranged side-by-side, with a larger diameter lumen in the centre and smaller diameter lumens to either side. Other suitable lumen cross section shapes could be used as discussed above. The lumens of tube 510 are arranged asymmetrically within the tube so as to be closer to the surface that is uppermost when the device is in use. In the embodiment shown in FIG. 14(e) tube 510 has a total width of approximately 1.49 mm to approximately 1.57 mm and a maximum height of approximately 200 microns. The central lumen has a diameter equal to approximately 60% of the maximum height of the tube, while the two side lumens have diameters equal to approximately 40% to approximately 50% of the diameter of the central lumen. The distance from the centre of the central lumen to the centre of each of the side lumens is equal to approximately 6 to approximately 7 times the diameters of the side lumens. The distance from the centre of each of the side lumens to the outer edge of the tube is equal to approximately 6.5 to approximately 7.5 times the diameters of the side lumens.

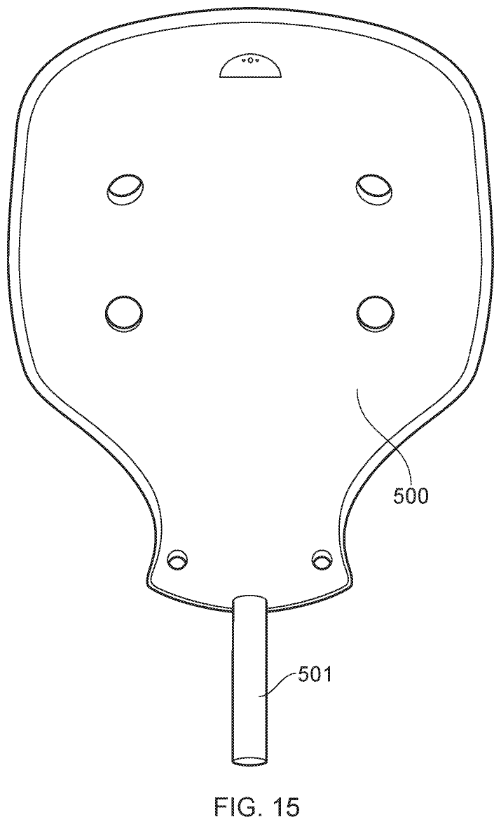

FIG. 15 shows a plate 500 for use with a multi-lumen tube 501. The multi-lumen tube is near identical to the tube 10 of the GDD 7 but for the absence of the wings 19. In all other respects the tube 10 and variants of it as described above may be used with the plate 500. The tube 501 opens in the underside of the plate 500 which can be secured to the patient's eye to stabilise the tube 501. The plate 500 may be used instead of the wings 19 of the GDD 7.

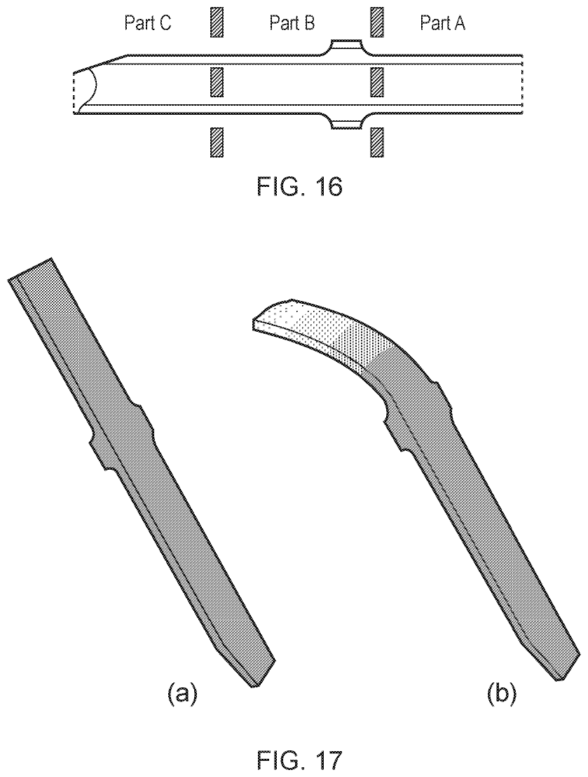

Any glaucoma device exerts locally stretching and compressive forces on the surrounding tissue when implanted in an human eye. It is therefore extremely important to minimise as much as possible these forces to prolong the life of the device and avoid any excessive scarring. There should be extra care on the device shape to achieve these goals. When inserted, the tube may be divided into three different zones (Parts A, B and C) with different requirements to optimise the interaction of the tube with the surrounding tissue as shown in FIG. 16.

Part A represents the part of the tube exposed to the conjunctival tissue. Part B is the section of the tube inside the scleral tunnel to maintain a seal between the anterior chamber and the subconjunctival tissue. Finally, Part C corresponds to the part of the tube inside the anterior chamber that will periodically flap due to blinking, eye saccades and head movements.

Part A—Minimising Conjunctival Tissue Stress

When the tube is in-situ, the back of the tube exposed to the conjunctival tissue (after the wings) is being deflected downwards to hold it in place along the surface of the eye. An example of the tube deflection is shown in FIG. 17 where the back of the GDD (posterior to the wings) is deflected downward by approximately 1.6 mm.

This will result in a contact zone with the tissue under pressure that needs to be minimised to reduce the trauma of the conjunctiva tissue as much as possible: Firstly, the indentation depth into the conjunctival tissue needs to be minimised to reduce the trauma on the tissue. Secondly, the maximum contact pressure for a fixed depth of penetration needs also to be minimised to reduce the stress on the tissue.

Tube indentation depth in the conjunctival tissue

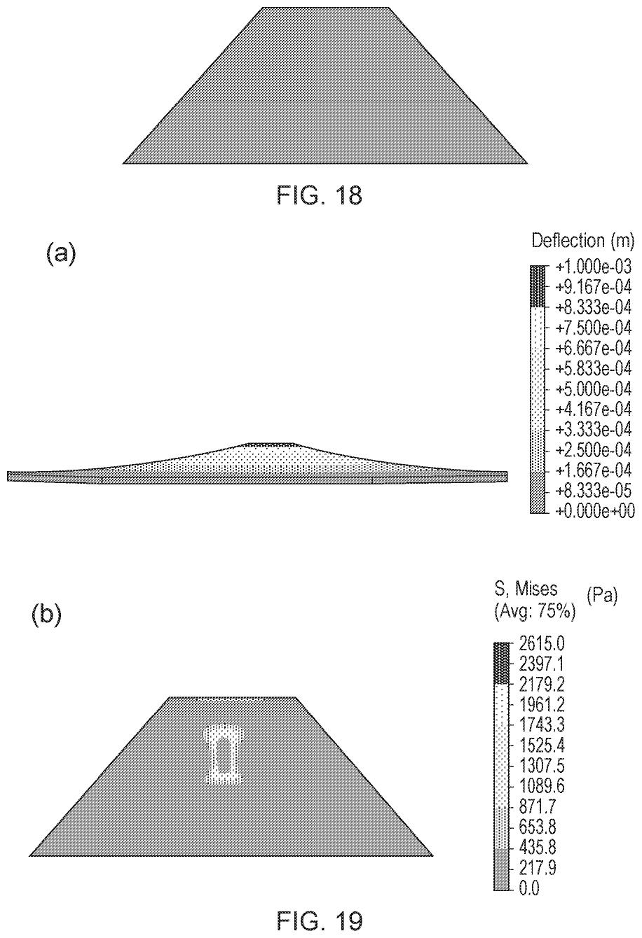

We used finite-element analysis to illustrate this point. We have modelled a 0.2 mm thick conjunctival flap as shown in FIG. 18.

Four indentation depths of respectively 1 mm depth by 1 mm wide, 0.5 mm depth by 0.5 mm wide, 0.25 mm depth by 0.25 mm wide and 0.125 mm depth by 0.125 mm wide were created locally. These indentation depths correspond to diameters of tubes resting on the sclera with the conjunctival tissue covering it. The length of the tube interacting with the conjunctival flap is 2.5 mm.

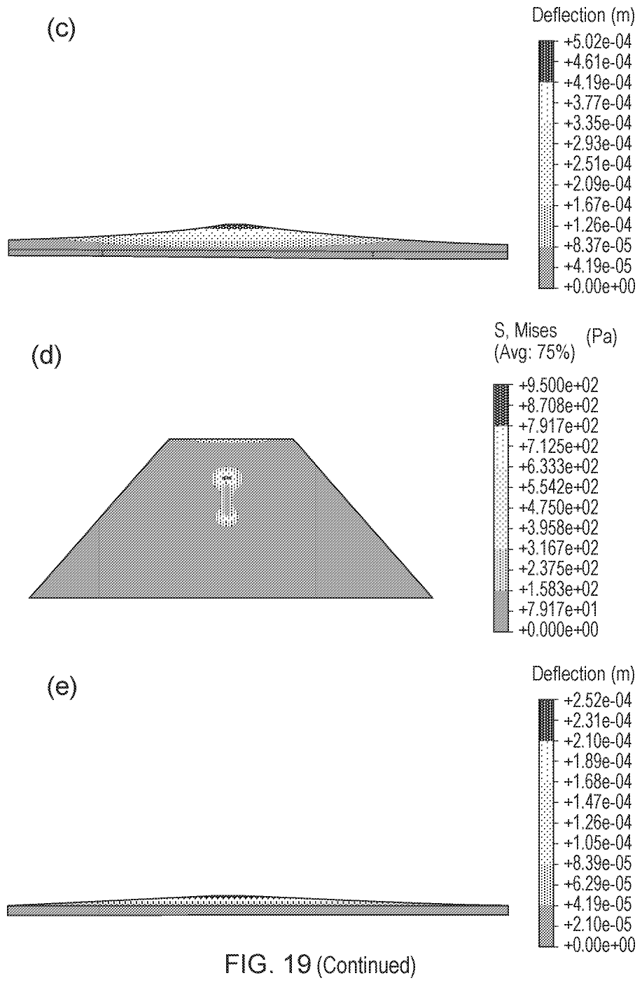

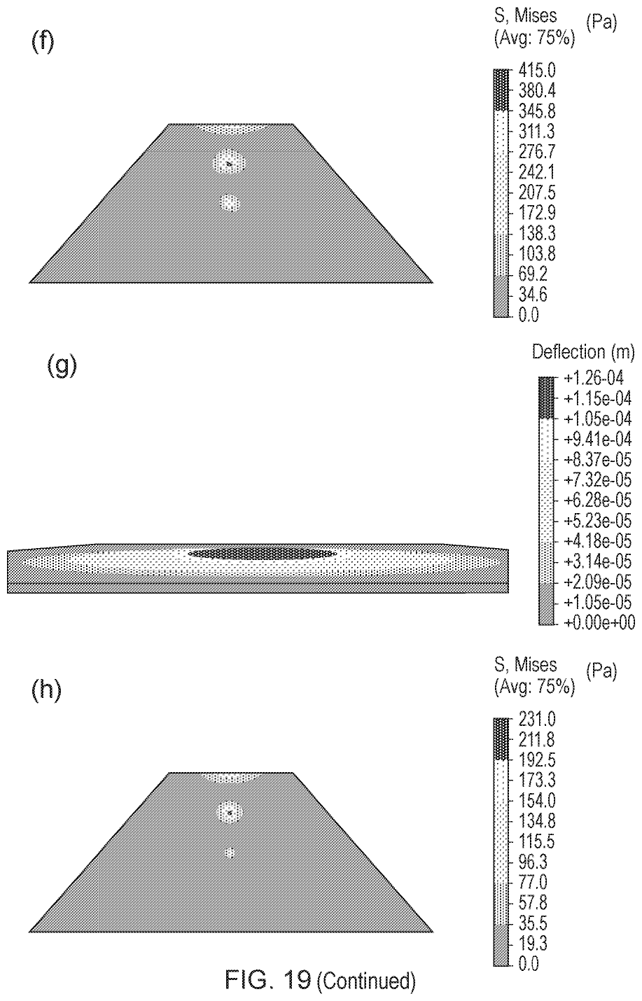

In FIG. 19, it can be seen that as the diameter of the tube decreases (a, c, e, g), the deflection of the conjunctival flap is reduced as expected. It can also be seen that the pyramidal shape of the tissue is greatly reduced as the tube external diameter is reduced. Indeed, this is reflected on the stress shown on the corresponding plots for 1 mm deflection in (b), 0.5 mm deflection in (d), 0.25 mm deflection in (f) and 0.125 mm deflection in (h). For example, reducing the tube diameter from 0.5 mm to 0.25 mm halves approximately the maximum Von Mises stress from 950 Pa to 415 Pa. It is therefore important to reduce the tube diameter to reduce the trauma on the conjunctiva. The first requirement of the tube is to have a small diameter to reduce the indentation into the conjunctival tissue.

Maximum contact pressure on the conjunctival tissue

The interaction between a tube and the subconjunctival tissue can be modelled as the interaction between a cylinder and a flat surface. Indeed, locally the curvature of the eyeball is between 1 to 2 orders of magnitude larger than the tube radius, the conjunctival tissue can be treated as a planar section. As shown in FIG. 20, the cylinder has a radius R and a length L, and is pushed with a force F towards the conjunctival tissue.

The contact surface has a width a defined by:

$$a = \sqrt{\frac{8 FR}{π {E}^{*}}} \qquad ( 1 )$$

with E* defined as:

$${E}^{*} = \frac{1 - {v}_{1}^{2}}{{E}_{1}} + \frac{1 - {v}_{2}^{2}}{{E}_{2}} \qquad ( 2 )$$

with E₁ and E₂, the elastic Young's modulii of the tube and the conjunctival tissue and v₁, v₂, the associated Poisson's ratios. Therefore, it can be seen that with increasing tube radius, the width of the surface contact increases as expected as the square root of the tube radius. The maximum pressure of contact is obtained at the centre of the surface contact and is defined as:

$${p}_{max} = \sqrt{\frac{{FE}^{*}}{π LR}} \qquad ( 3 )$$

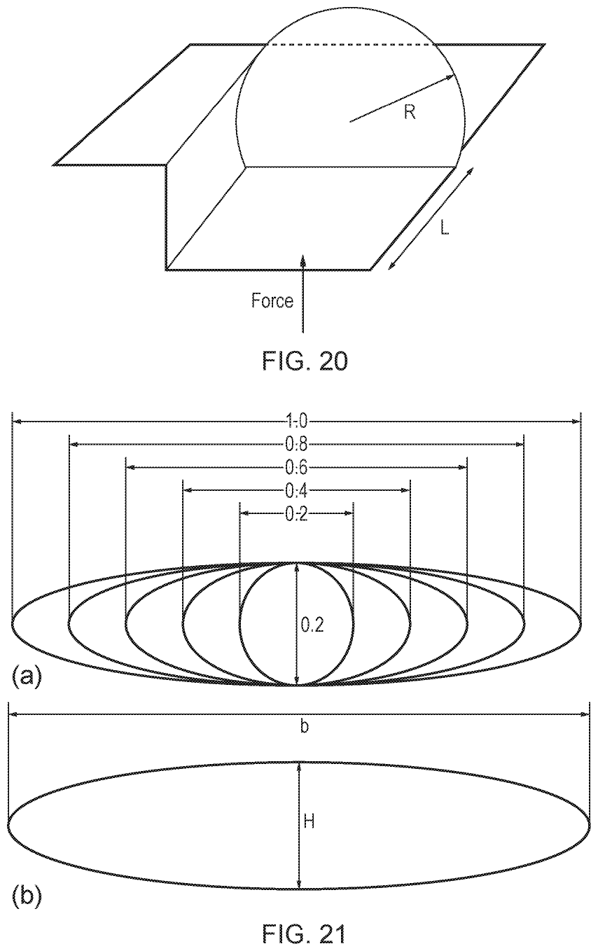

The maximum contact pressure is proportional to \sqrt{1/R} and can be reduced by increasing the radius of the tube for a fixed indentation. Therefore, it is important to increase the radius of the tube to decrease the maximum contact pressure exerted by the tube on the conjunctiva. The only way to increase the contact area of the tube with the tissue while keeping a fixed indentation length is by adopting an elliptical shape to increase the width of the tube while keeping its height constant as shown in FIG. 21(a). In other words, it consists in increasing the eccentricity of the tube, e, which is defined as:

$$e = \sqrt{1 - \frac{{H}^{2}}{{b}^{2}}} \qquad ( 4 )$$

with H being the height of the tube (or twice the semiminor axe), and b, the width of the tube (or twice the semimajor axe) as shown in FIG. 21(b).

It is therefore important to increase the eccentricity of the tube to reduce the maximum contact pressure between the tube and the conjunctival tissue. The GDD described herein preferably has an eccentricity of approximately 0.98.

Part B: Minimising Scleral Incision Seal

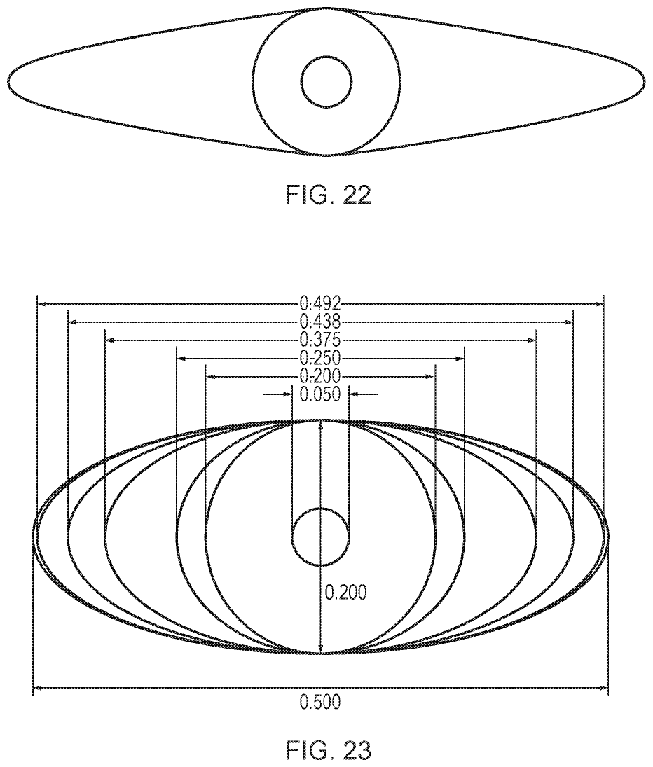

Each tube in glaucoma surgery must be inserted through an incision that connects the anterior chamber to other part of the eye: the subconjunctival or suprachoroidal space. However, incision in the tissue is usually made using a knife and this gives a straight cut. Therefore, a circular tube placed in a horizontal cut will result in the tissue being stretched upwards, resulting in leaks around the outside of the tube circular tube as the incision tends to be mainly elliptical as shown in FIG. 22.

In first approximation, the incision can be modelled with an elliptical shape. We simulated a flow of 2 μl/min going through a 3 mm long tube with a lumen of 50 μm of increasing eccentricity inserted into an incision of 3 mm length (0.2 mm high and 0.5 mm wide) and recorded the pressure drop across the set-up. We chose 5 different shapes of tube with an height of 0.2 mm and width of 0.2 mm (e=0), 0.25 mm (e=0.6), 0.375 mm (e=0.85), 0.438 mm (e=0.89) and 0.492 mm (e=0.91) as shown in FIG. 23. All tubes have a lumen diameter of 0.05 mm.

Monitoring the pressure drop for all configurations gives us an indication of leakage. The pressure drop should be around 5-6 mmHg if the incision is sealed properly as the flow should only be going through the tube, any lower value means there is leakage. We found that for the first three configuration (b=0.2, 0.25 and 0.375 mm), the pressure drop through the set up was well below 1 mmHg, showing important leakage. However, for the fourth configuration (0.438 mm), the pressure drop was around 3 mmHg, which is approximately half of the correct value, denoting that flow is going through the tube as well as the space created by the incision. When the width of the tube was 0.492 mm (fifth configuration), the pressure drop was correct at around 5-6 mmHg, showing that the flow was going predominantly through the tube.

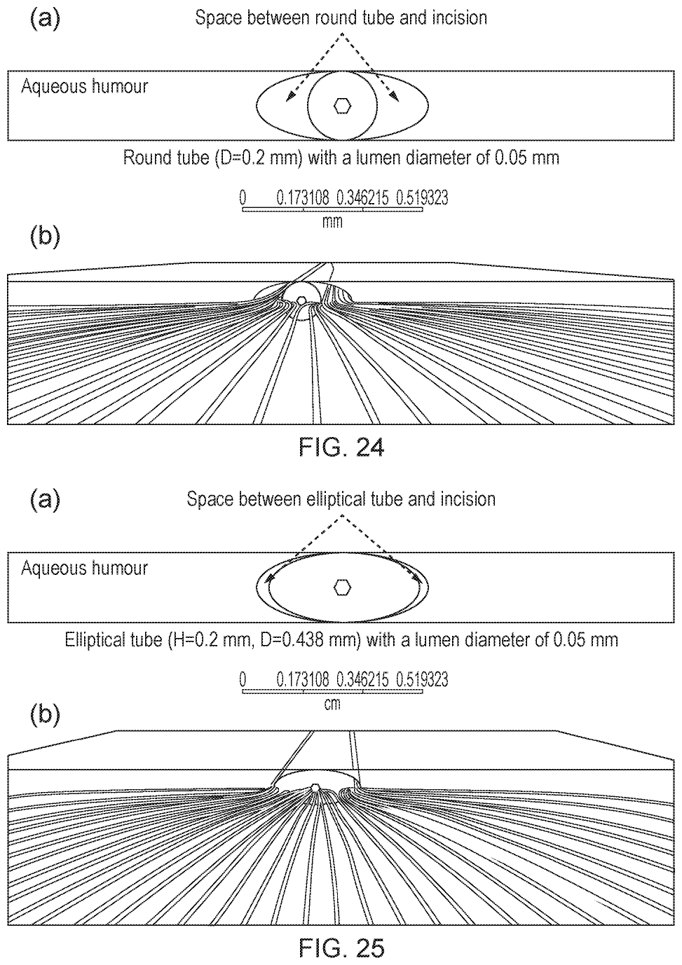

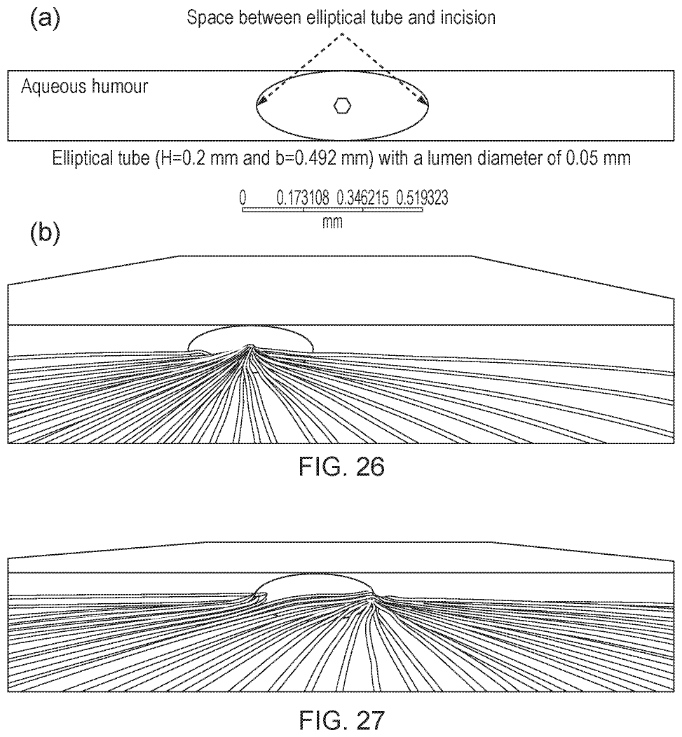

The pathlines of particles seeded in the anterior chamber are plotted to show: (i) where there is important leakage (FIG. 24); (ii) where the flow goes through the tube and the space created by the incision (FIG. 25); (iii) where the flow goes mainly through the tube (FIG. 26).

We conducted additional simulations for the configuration shown in FIG. 26, where we plugged the lumen and calculated the pressure drop required to push aqueous humour fluid (identical viscosity) at a flow rate at 2 μl/min through the gap in the incision between the tube and the tissue as shown in FIG. 27. The pressure obtained was in excess of 400 mmHg with the lumens of the tube closed. This demonstrates that the flow tends to go preferably through an open lumen which is the path of least resistance, hence there was minimal leakage on the outside of the tube for this eccentricity.

These simulations hold true for any incision dimension with the same outcome. Elliptical tubes inserted into incisions tend to reduce leakage due to being similar shape to the incision which has been made.

Part C: Minimising Tube Flapping in the Anterior Chamber

Flapping of an end of a tube in the anterior chamber of the eye can cause trauma to the corneal epithelium. The elliptical shape of the tube defined in the previous section will reduce the deflection of the tube in the anterior chamber for a fixed force. If we simplified the tube as being held in place at the wings at the limbus section, the potential displacement of the tube in the anterior part of the chamber can be modelled as a cantilever beam held in place at the wing section. In that case, the small deflection of cantilever beams A is calculated as:

$$Δ = \frac{{L}^{3} F}{3 {E}_{1} I} \qquad ( 5 )$$

with F: the force exerted on the tube, E₁: the Young's Modulus of the tube material, I: the second moment of area and L: the length of the tube being displaced. The parameter I characterises the section geometry of the tube in relation to a given axis, depending on the direction of the deflection. The higher the value of I, the lower the deflection of the tube for a given force. The tube in the anterior chamber can flap in any direction. We will concentrate on the main two directions and any other direction will be a combination of these two directions.

First, we look at the displacement of the tube normal to the surface of the cornea. In that direction, the second moment of area for a cylinder:

$${I}_{ex} = \frac{\text{?}}{\text{?}} . \\ \text{?} \text{indicates text missing or illegible when filed}$$

with D being the outer diameter. For an elliptical shape, the second moment of area is given by

$${I}_{ex} = \frac{\text{?}}{\text{?}} , \\ \text{?} \text{indicates text missing or illegible when filed}$$

with H, the height of the oval and b, the width of the tube as shown in FIG. 21(b). For a similar force, Young's Modulus and length, the ratio of the displacement (Equation 5) of the cylindrical tube versus the elliptical tube can be rewritten solely using the second moment of area I. We assume that the cylindrical and elliptical tube have the same height (D=H). In that case, the ratio of deflection of a circular tube over an elliptical tube for a tube of identical length, L, Young's Modulus, E, and force, F, can be rewritten entirely from Equation 5 as:

$$\begin{matrix}\frac{\text{?}}{\text{?}} = \frac{b}{H} & ( 6 ) \\ \text{?} \text{indicates text missing or illegible when filed} & \end{matrix}$$

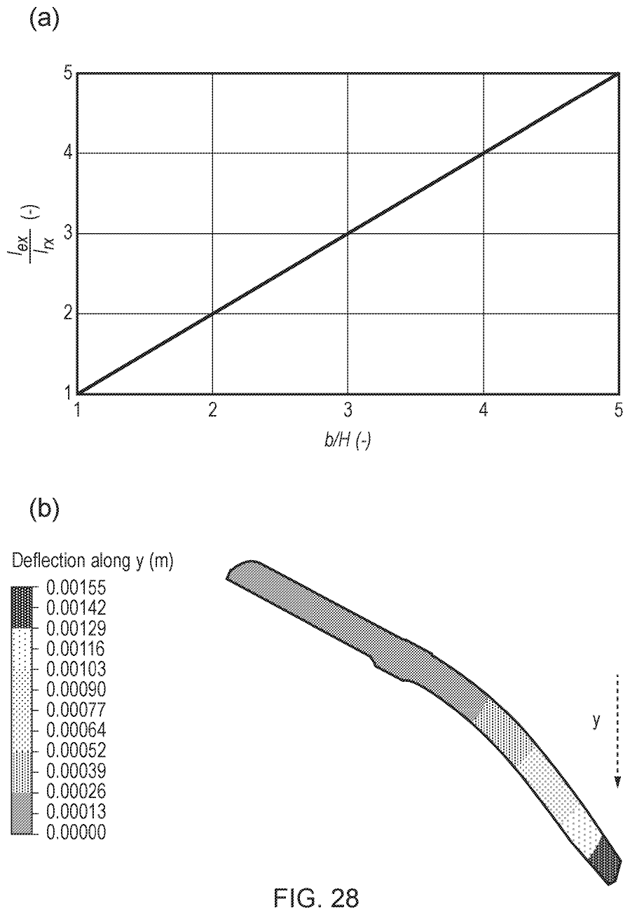

The ratio on deflection only depends on two factors: the width of the elliptical tube over the height or diameter of the circular tube. For example, if the tube has a diameter D=0.2 mm with D=H and the elliptical tube has a width of 1 mm, we have Iₑₓ/Iᵣₓ=5. It means that the GDD described herein reduces the deflection of the elliptical tube normal to the cornea by a factor of 5 compared to a circular tube (D=0.2 mm) according to equation (5) for a fixed force. Further characterisation of this ratio is shown in FIG. 28(a) and an illustration of the deflection from the GDD is shown in FIG. 28(b).

Secondly, lateral displacements of the tube should also be reduced to avoid any rubbing of the epithelium of the cornea by the tube. Indeed, a circular tube anchored at one point can move in any direction (normal and parallel to the cornea) with the same force. However, for an elliptical tube, this is not the case. We minimise any lateral displacement by increasing the second moment of area relative to parallel displacement. The force described in Equation (5) is still valid and we simply need to modify the second moment of area to reflect lateral displacement. For a circular tube, the second moment of area is unchanged and equal to

$$\text{?} = \frac{\text{?}}{\text{?}} . \\ \text{?} \text{indicates text missing or illegible when filed}$$

For the elliptical tube, it becomes

$$\text{?} = \frac{\text{?}}{\text{?}} . \\ \text{?} \text{indicates text missing or illegible when filed}$$

Therefore, if we assume D=H, we now have for lateral displacement:

$$\begin{matrix}\frac{\text{?}}{\text{?}} = \frac{{b}^{3}}{{H}^{3}} & ( 7 ) \\ \text{?} \text{indicates text missing or illegible when filed} & \end{matrix}$$

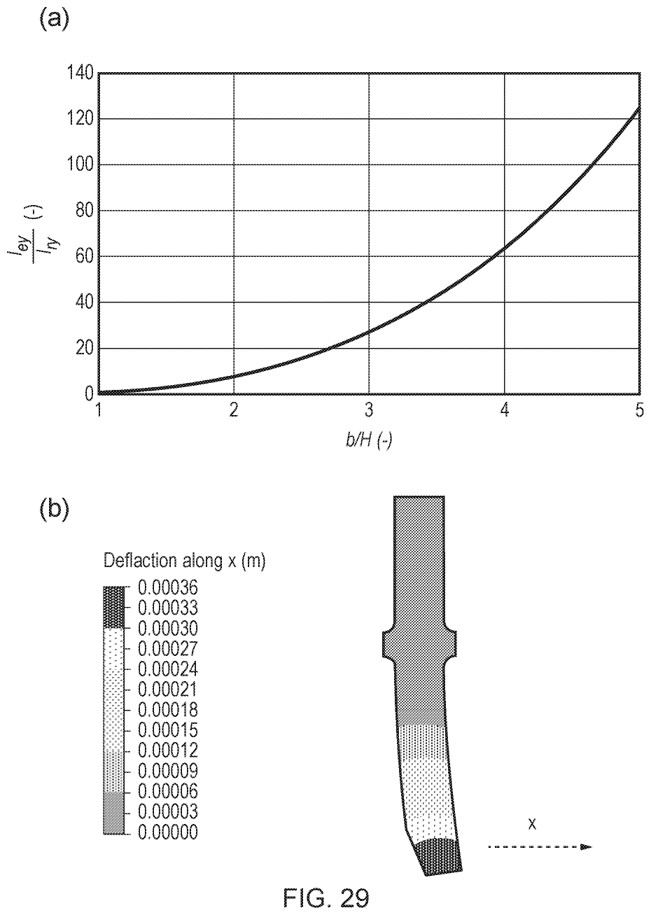

The ratio is plotted in FIG. 29(a) with an illustration of the deflection from the GDD shown in FIG. 29(b).

This ratio is proportional to b³/H³ and is equal to 125 when b=1 mm and H=0.2 mm. The GDD reduces the deflection of the elliptical tube parallel to the cornea by a factor of 125 compared to a circular tube (D=0.2 mm) according to equation (5) for a given force.

In conclusion, adopting an elliptical shape significantly reduces the flapping of the tube in any direction which can damage the corneal epithelium layer over time.

Part A-B-C: Minimising Tube Internal Stress

General Tube Bending

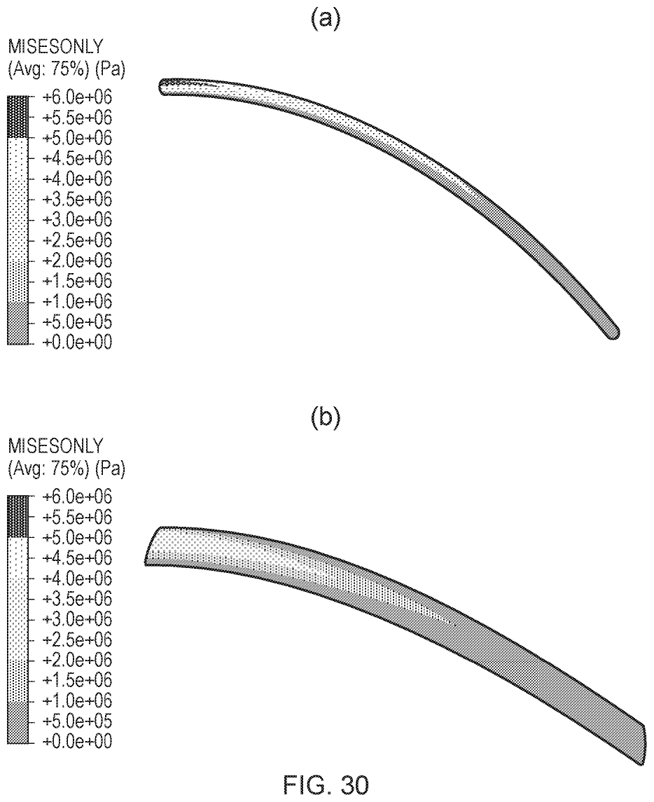

It is important to minimise the stress that the tube is under when implanted. Indeed, the tube is under constant stress as it is bent when implanted. This is a different requirement from minimising the surface contact pressure and concerns the internal stress that the tube undergoes when implanted. The shape is instrumental in reducing internal stress. In FIG. 30, it can be seen that for the same deflection force, the internal stress of the elliptical shape of an example of the GDD of eccentricity 0.98 (H=0.2 mm and b=1 mm) is reduced to a third of that of a circular tube of the same height (D=0.2 mm). This illustrates that an elliptical shape also reduces the internal stress of the tube and could therefore prolong the life of the device since it undergoes relatively lower stress.

Specific Tube Bending through Incision

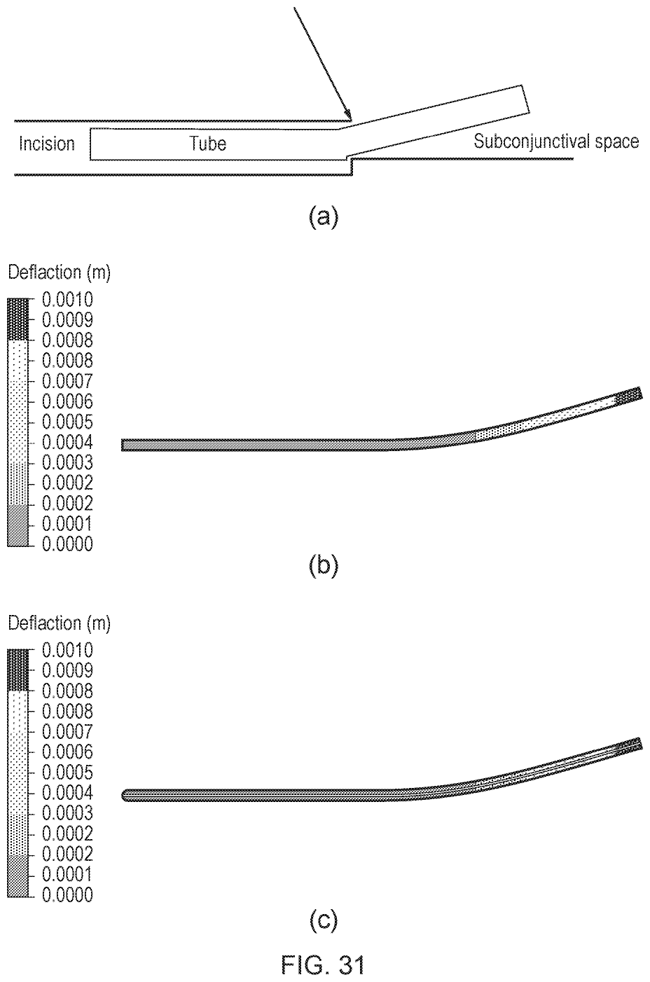

When the tube exits from the incision into the subconjunctival space, the tube is under stress at the point of exit shown by an arrow in FIG. 31(a). It is important to reduce the internal stress of the tube as it may remain in that position for decades. Two Finite-Element Analysis have been conducted, with a circular tube of diameter 0.2 mm and an elliptical tube of height 0.2 mm and width 1 mm (eccentricity of 0.98 similar to the GDD), and an upward deflection of 1 mm as shown in FIG. 31(b) and (c), respectively.

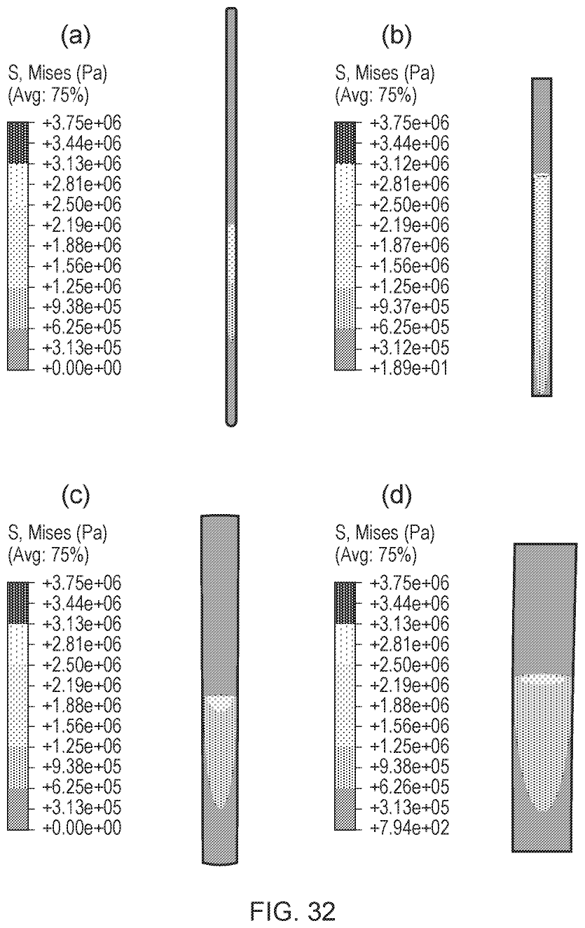

The corresponding tube internal stresses of the deflections shown in FIG. 31 are plotted in FIG. 32. It can be seen that the maximum internal stress of the tube is reduced by approximately 25% from 3.75 MPa to around 2.8 MPa when a circular tube of 0.2 mm diameter is compared to an elliptical tube of height 0.2 mm and width 1 mm, similar to the GDD. Therefore, an elliptical shape reduces internal stress of the tube when it exits the incision into the subconjunctival shape.

Although the invention has been described above with reference to one or more preferred embodiments, it will be appreciated that various changes or modifications may be made without departing from the scope of the invention as defined in the appended claims.

1. A drainage device for use in an eye to drain aqueous humour so as to reduce intraocular pressure, the device comprising a flexible multi-lumen tube having a first end, a second end opposite the first end, a longitudinal axis through the first end and the second end, a plurality of lumen extending between the first end and the second end, and an outer surface extending between the first end and the second end, wherein a cross-section perpendicular to the longitudinal axis has a non-circular shape at the outer surface with an aspect ratio of at least 3:1. width:height and/or an eccentricity between approximately 0.6 and approximately 0.98.

2. A drainage device according to claim 1, wherein the cross-section shape at the outer surface is an ellipse.

3. A drainage device according to claim 1, wherein the multi-lumen tube is anisotropic in bending about two axes each perpendicular to the longitudinal axis.

4. A drainage device according to claim 3, wherein the multi-lumen tube has a width and a height, and the plurality of lumen are spaced in the width dimension, and the tube has greater bending flexibility in a plane including the height dimension than in a plane including the width dimension.

5. A drainage device for use in an eye to drain aqueous humour so as to reduce intraocular pressure, the device comprising a multi-lumen tube having a first end, a second end opposite the first end, and a plurality of lumen extending between the first end and the second end, wherein at least one of the lumen is sealed at the first end, and further comprising at least one aperture open in said at least one of the lumen through a sidewall of the tube and located along the length of the tube between the first end and the second end, wherein the at least one aperture fluidly connects the second end of the tube to outside the tube through said lumen.

6. A drainage device according to claim 5, wherein a distance from the second end to the aperture is selected to provide predetermined resistance to fluid flow through the device.

7. A drainage device according to claim 5, further comprising a plurality of the apertures.

8. A draining device according to claim 7, wherein a plurality of the lumen each have at least one of the apertures discretely through the sidewall.

9. A drainage device according to claim 7, wherein said at least one of the lumen has a plurality of the apertures spaced along the length of the tube.

10. A drainage device according to claim 1, wherein said at least one of the lumen has an internal diameter selected to provide predetermined resistance to fluid flow through the device.

11. A drainage device according to claim 1, wherein the tube is flexible.

12. A drainage device according to claim 1, wherein each lumen has a diameter of between approximately 40 microns to approximately 200 microns.

13. A drainage device according to claim 1, wherein each lumen has a substantially constant cross section along the length of the tube.

14. A drainage device according to claim 1, wherein the tube length is between approximately 5 mm to approximately 30 mm.

15. A drainage device according to claim 1, wherein the tube width is between approximately 0.5 mm to approximately 3 mm.

16. A drainage device according to claim 1, wherein a maximum height of the tube is approximately 500 microns or less.

17. A drainage device according to claim 1, wherein two or more of the lumen have different internal diameters.

18. A drainage device according to claim 1, wherein one or more of the lumen have a substantially circular cross section.

19. A drainage device according to claim 1, wherein the tube includes biocompatible and/or biostable material.

20. A drainage device according to claim 1, wherein the tube includes at least one of plastics material and silicone.

21. A drainage device according to claim 1, wherein a sidewall of the tube has a thickness of between approximately 5 microns to approximately 200 microns.

22. A drainage device according to claim 1, wherein the tube includes transparent or translucent material.

23. A drainage device according to claim 1, wherein each lumen is valveless.

24. A drainage device according to claim 1, wherein the first end of the tube has a bevelled edge.

25. A drainage device according to claim 1, further comprising generally planar extensions projecting from the tube intermediate the first and second ends.

26. (canceled)

27. A drainage device according to claim 1, further comprising a plate adapted to locate on the eye, and wherein the first end of the multi-lumen tube opens in the underside of the plate.

28. A method of manufacturing a drainage device for use in an eye to drain aqueous humour so as to reduce intraocular pressure, comprising providing a multi-lumen tube having a first end, a second end opposite the first end, a plurality of lumen extending between the first end and the second end, and adjusting a flow through the multi-lumen tube by forming at least one aperture open in one of the lumen through a wall of the tube and/or sealing at least one aperture open in one of the lumen.

29. A method according to claim 28, wherein forming the aperture comprises forming the aperture through a sidewall of the tube.

30. A method according to claim 28, wherein at least one of the lumen is sealed at the first end to provide an end wall, and forming the aperture comprises forming the aperture through the end wall of the sealed first end.

31. A method according to claim 30, wherein sealing the aperture comprises either closing an open first end of the lumen or closing an aperture through a sidewall of the tube.

32. A method according to claim 28, wherein the aperture is formed by laser cutting.

33. A method according to claim 28, wherein the multi-lumen tube is made by extrusion, drawing or injection moulding, wherein extrusion comprises extruding a multi-lumen preform through a die, optionally stretching the preform in the longitudinal direction to reduce the lumen diameter and cutting to a desired tube length.

34. A method according to claim 28, for manufacturing a drainage device.

35. A method for treating glaucoma or controlling intraocular pressure in a patient's eye with a drainage device, wherein the drainage device comprises a flexible multi-lumen tube having a first end, a second end opposite the first end, a longitudinal axis through the first end and the second end, a plurality of lumen extending between the first end and the second end, and an outer surface extending between the first end and the second end, wherein a cross-section perpendicular to the longitudinal axis has a non-circular shape at the outer surface with an aspect ratio of at least 3:1 width:height and/or an eccentricity between approximately 0.6 and approximately 0.98, the method comprising positioning the first end of the drainage device in the anterior chamber of the patient's eye, and positioning the second end of the drainage device in the subconjunctival space of the patient's eye.

36. A method according to claim 35, further comprising opening one or more apertures in one or more of the lumens to control the flow rate of aqueous humour through the drainage device.

37. A method for preparing a drainage device for surgery,
the drainage device comprising a multi-lumen tube having a first end a second end opposite the first end, a plurality of lumen extending between the first end and the second end, wherein at least one of the lumen is sealed at the first end, and further comprising at least one aperture open in said at least one of the lumen through a sidewall of the tube and located along the length of the tube between the first end and the second end, wherein the at least one aperture fluidly connects the second end of the tube to outside the tube through said lumen,
the method comprising: comparing an intraocular pressure measurement obtained from a patient with a threshold to calculate the required drop in intraocular pressure, and opening the at least one aperture in the at least one lumen to control the flow rate of aqueous humour through the drainage device and provide the required drop in intraocular pressure.

38. A kit comprising a drainage device according to claim 1 and complimentary forceps and/or blade and/or inserter.

39. A drainage device according to claim 1, further comprising at least one aperture open in at least one of the lumen through a sidewall of the tube and located along the length of the tube between the first end and the second end, wherein the at least one aperture fluidly connects the second end of the tube to outside the tube through said lumen.

40. A drainage device according to claim 39, further comprising generally planar extensions projecting from the tube intermediate the first and second ends.

41. A drainage device according to claim 40, wherein the at least one aperture is located between the generally planar extensions and the first end.
